(19) United States
(12) Patent Application Publication
(10) Pub. No.: US 2025/0104843 A1
(43) Pub. Date: Mar. 27, 2025

(54) IMAGE MANAGEMENT METHOD AND IMAGE MANAGEMENT DEVICE

(71) Applicant: CASIO COMPUTER CO., LTD., Tokyo (JP)

(72) Inventor: Hiroyuki TSUKADA, Tokyo (JP)

(73) Assignee: CASIO COMPUTER CO., LTD., Tokyo (JP)

(21) Appl. No.: 18/887,141

(22) Filed: Sep. 17, 2024

(30) Foreign Application Priority Data
Sep. 21, 2023 (JP) 2023-154085

(51) Int. Cl. G16H 30/20

(52) U.S. Cl. CPC G16H 30/20

(57) ABSTRACT

Disclosed is an image management method executed by a computer, the method including: setting an imaging apparatus group including a plurality of imaging apparatuses in response to a user's operation of selecting the imaging apparatuses; registering identification information capable of identifying a subject in association with the imaging apparatus group; acquiring a plurality of images captured by respective ones of the plurality of imaging apparatuses included in the imaging apparatus group; and linking the acquired images with the identification information.

### REFERENCE TO RELATED APPLICATIONS

This application is based upon and claims the benefit of priority from the prior Japanese Patent Application No. 2023-154085, filed on Sep. 21, 2023, the entire contents of which are incorporated herein by reference.

### TECHNICAL FIELD

The present disclosure relates to an image management method and an image management device.

### DESCRIPTION OF RELATED ART

Conventionally, there is known a technique for performing imaging for a plurality of sites of a subject (such as a patient) using a single imaging apparatus for capturing images (for example, JP-A-2006-263223).

### SUMMARY OF THE INVENTION

In order to solve the above problems, there is provided an image management method executed by a computer, the method including: setting an imaging apparatus group including a plurality of imaging apparatuses in response to a user's operation of selecting the imaging apparatuses; registering identification information capable of identifying a subject in association with the imaging apparatus group; acquiring a plurality of images captured by respective ones of the plurality of imaging apparatuses included in the imaging apparatus group; and linking the acquired images with the identification information.

### BRIEF DESCRIPTION OF DRAWINGS

The accompanying drawings are not intended as a definition of the limits of the invention but illustrate embodiments of the invention, and together with the general description given above and the detailed description of the embodiments given below, serve to explain the principles of the invention, wherein:

FIG. 1 is a block diagram showing a configuration of a medical image management system;

FIG. 2 is a side view of a first camera;

FIG. 3 is a side view of a second camera;

FIG. 4 is a block diagram showing a main functional configuration of the first camera;

FIG. 5 is a block diagram showing a main functional configuration of the second camera;

FIG. 6 is a block diagram showing a main functional configuration of a medical image management device;

FIG. 7 is a diagram showing a camera group setting screen;

FIG. 8 is a diagram showing a patient ID input screen for inputting a patient ID;

FIG. 9 is a diagram showing camera group management data in a state in which a patient ID is associated;

FIG. 10 is a diagram illustrating operations for transmitting medical images from the first camera and the second camera;

FIG. 11 is a diagram showing the contents of image management data;

FIG. 12 is a flowchart showing a control procedure of a camera group setting process;

FIG. 13 is a flowchart showing a control procedure of a medical image registration process;

FIG. 14 is a diagram showing operations for transmitting medical images according to a modification example;

FIG. 15 is a flowchart showing a control procedure of a medical image registration process according to the modification example;

FIG. 16 is a diagram showing the contents of image management data according to the modification example;

FIG. 17 is a diagram showing a camera group setting screen; and

FIG. 18 is a flowchart showing a control procedure of a medical image registration process according to a second embodiment.

### DETAILED DESCRIPTION

Hereinafter, an embodiment of the present disclosure will be described with reference to the drawings.

### First Embodiment

### Configuration of Medical Image Management System

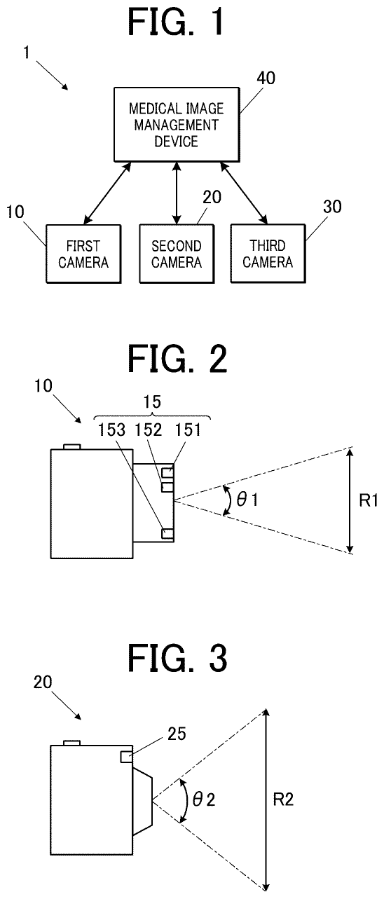

FIG. 1 is a block diagram showing the configuration of a medical image management system 1. The medical image management system 1 (image management system) includes a first camera 10 (first imaging apparatus), a second camera 20 (second imaging apparatus), a third camera 30, and a medical image management device 40 (image management device). The medical image management system 1 is installed in, for example, a medical institution such as a hospital. The first camera 10, the second camera 20, and the third camera 30 are medical cameras used by a doctor to examine a patient (subject). The first camera 10, the second camera 20, and the third camera 30 are operated by the doctor to capture a site such as an affected area of the patient and generate image data of a medical image (image). The number of cameras included in the medical image management system 1 is not limited to three, and may be two, four or more.

The medical image management device 40 is connected to the first camera 10, the second camera 20, and the third camera 30 wirelessly or via a wire so as to be able to perform data communication. In FIG. 1, the medical image management device 40 is directly connected to the first camera 10, the second camera 20, and the third camera 30, but the connection is not limited to this, and the medical image management device 40 may be indirectly connected to each camera. For example, in a configuration in which a medical image management device 40 is communicatively connected to a wireless LAN access point, the first camera 10, the second camera 20 and the third camera 30 may be configured to transmit and receive data to and from the medical image management device 40 via wireless communication with the access point. The medical image management device 40 manages image data of medical images captured by the first camera 10, the second camera 20 and the third camera 30 and transmitted to the medical image management device 40. Hereinafter, “image data of medical image(s)” will also be referred to simply as “medical image(s)”.

Next, the configurations of the first camera 10 and the second camera 20 will be described. The configuration of the third camera 30 is arbitrary and may be the same as the first camera 10 or the second camera 20 or may be different from the configuration of either the first camera 10 or the second camera 20.

FIG. 2 is a side view of the first camera 10.

FIG. 3 is a side view of the second camera 20.

As shown in FIG. 2, the first camera 10 is capable of capturing an angle of view θ1 in the up-down direction (vertical direction) when the first camera 10 is held horizontally.

On the other hand, as shown in FIG. 3, the second camera 20 is capable of capturing an angle of view θ2 in the vertical direction that is larger than the angle of view θ1. In other words, the focal length of the second camera 20 is shorter than the focal length of the first camera 10.

Therefore, the first camera 10 has a first imaging range R1 shown in FIG. 2, and the second camera 20 has a second imaging range R2 shown in FIG. 3, and the vertical length of the second imaging range R2 is longer than the vertical length of the first imaging range R1 (first imaging range).

Although not shown in FIGS. 2 and 3, when the first camera 10 and the second camera 20 are held horizontally, the angle of view of the second camera 20 is also larger than the angle of view of the first camera 10 in the left-right direction (horizontal direction). Therefore, the horizontal length of the second imaging range R2 of the second camera 20 is longer than the horizontal length of the first imaging range R1 of the first camera 10. From the above, the second imaging range R2 of the second camera 20 is greater than the first imaging range R1 of the first camera 10.

The first camera 10, which has a relatively small angle of view compared to the second camera 20, is preferably used for imaging of an affected area (first site) inside the patient's body. In the present embodiment, the first camera 10 is used for imaging of the internal vulva (for example, cervix) of a female patient as a first site. In other words, the first camera 10 is used for imaging in colposcopy.

On the other hand, the first camera 10 has a small angle of view and is therefore not suitable for imaging of a site (for example, the vulva) covering a certain range or more on the surface of the patient's body. Therefore, the site on the surface of the patient's body is captured by the second camera 20, which has a relatively large angle of view. In the present embodiment, the second camera 20 is used for imaging of the patient's vulva as a second site different from the first site.

As shown in FIG. 2, the first camera 10 also includes a light emitting section 15 for illuminating a site that is a target of imaging during imaging. The light emitting section 15 has a normal light 151, a green light 152 and a polarized light 153, one of which is selected for use by the operator.

The normal light 151 emits normal white light.

The green light 152 emits green light mainly contains visible light in the green wavelength range. After applying acetic acid to the cervix, imaging is performed using the green light 152 as illumination, thereby making it possible to capture a medical image that highlights the blood vessels of the cervix.

The polarized light 153 emits linearly polarized light. The polarized light 153 may be a light that emits light with a random polarization direction and is equipped with a polarizer that transmits linearly polarized light. By using the polarized light 153 as illumination and capturing an image through a polarizing filter, surface reflection on mucous membranes and the like can be suppressed.

The brightness of the normal light 151, the green light 152 and the polarized light 153 is higher than the brightness of the light emitting section 25 of the second camera 20. The first camera 10 having such a high-brightness light emitting section 15 is suitable for imaging of the internal vulva, which is difficult to reach with external light.

As shown in FIG. 3, the second camera 20 includes a light emitting section 25 for illuminating a site that is a target of imaging during imaging. The light emitting section 25 emits normal white light, similarly to the normal light 151 of the first camera 10. However, if there is a sufficient amount of external light during imaging, the light emitting section 25 does not need to be used.

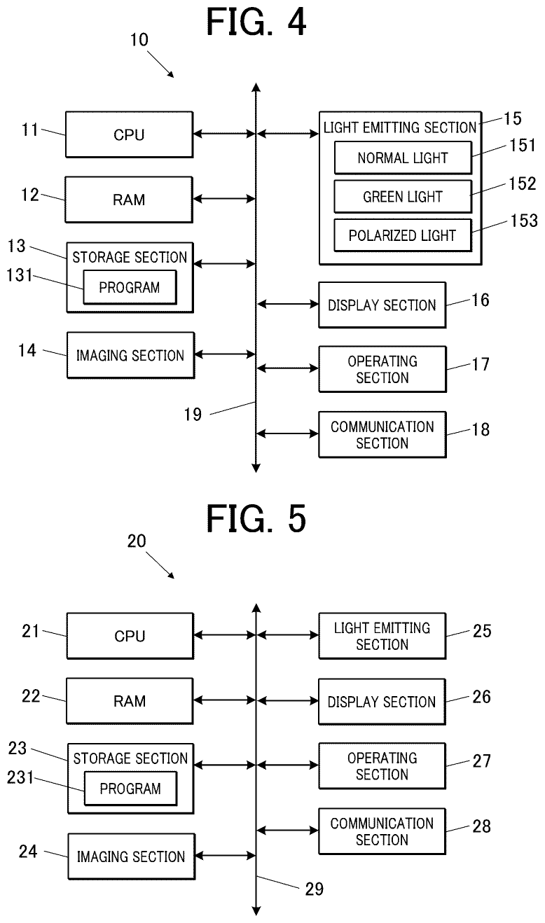

Next, the functional configurations of the first camera 10, the second camera 20, and the medical image management device 40 will be described with reference to FIGS. 4 to 6.

FIG. 4 is a block diagram showing the main functional configuration of the first camera 10.

The first camera 10 includes a CPU 11 (Central Processing Unit), a RAM 12 (Random Access Memory), a storage section 13, an imaging section 14, a light emitting section 15, a display section 16, an operating section 17, a communication section 18, and the like, and each of these sections is connected by a bus 19.

The CPU 11 is a processor that controls the operation of the first camera 10 by reading and executing a program 131 stored in the storage section 13 and performing various arithmetic processing. In addition, the first camera 10 may have a plurality of processors (for example, a plurality of CPUs), and the plurality of processes executed by the CPU 11 of the present embodiment may be executed by the plurality of processors. In this case, the plurality of processors may participate in a common process, or the plurality of processors may independently execute different processes in parallel. During imaging of a patient, the CPU 11 causes the light emitting section 15 to emit light in accordance with settings and user operation and performs imaging using the imaging section 14 in accordance with user operation that instructs imaging, thereby generating image data of a medical imaging the patient. Furthermore, the CPU 11 can add, as supplementary information, a patient ID (identification information) from which the captured patient can be identified to the generated medical image. The patient ID may be included, for example, in the EXIF (Exchangeable image file format) information of the medical image. This patient ID is, for example, input by a doctor who operates the first camera 10 using the operating section 17 prior to imaging by the first camera 10. The EXIF information may include information such as the date and time of imaging, identification information such as the serial number of the camera that captured the image, various imaging conditions (shutter speed, aperture, ISO sensitivity, and the like), and which of the three lights in the light emitting section 15 was used.

The RAM 12 provides a working memory space for the CPU 11 and stores temporary data.

The storage section 13 is a non-transitory recording medium that can be read by the CPU 11 as a computer and is configured from a non-volatile storage device such as a flash memory. The storage section 13 stores a program 131 executed by the CPU 11, various data, and the like. The program 131 is stored in the storage section 13 in the form of computer-readable program code.

The imaging section 14 includes an optical system that forms an image from incident light, an imaging element that detects the incident light from which the image is formed by the optical system, and an image processing section that generates image data based on the detection signal output from the imaging element. The optical system can be composed of, for example, a mirror and a lens group. The imaging element is not particularly limited as long as it is capable of photoelectrically converting incident light, and may be, for example, a CCD (Charge Coupled Device) or a CMOS (Complementary Metal Oxide Semiconductor). The image processing section amplifies and A/D converts the detection signal from the imaging element to generate image data of the captured subject. The image processing section may also perform various types of image processing, such as shading correction and color correction, on the generated image data.

The light emitting section 15 emits light for illuminating a subject in the direction of imaging by the imaging section 14. As mentioned above, the light emitting section 15 comprises a normal light 151, a green light 152 and a polarized light 153. The on/off of light emission by the light emitting section 15 is controlled by a control signal sent from the CPU 11.

The display section 16 includes a display device such as a liquid crystal display. In accordance with the image data and control signals transmitted from the CPU 11, the display section 16 displays the captured image as a live view image in real time on the display screen of the display device, plays back and displays the captured image, and displays various operation icons.

The operating section 17 has a touch panel provided over the display screen, and operation buttons provided on the housing of the first camera 10. The operating section 17 detects touches by fingers or the like on the touch panel and operations on the operation buttons, and outputs operation signals according to the detection results to the CPU 11.

The communication section 18 is composed of, for example, an antenna, a modulation/demodulation circuit, a signal processing circuit, and the like. The communication section 18 transmits and receives data to and from the medical image management device 40 or a wireless LAN access point or the like in accordance with a predetermined communication standard.

FIG. 5 is a block diagram showing the main functional configuration of the second camera 20.

The second camera 20 includes a CPU 21, a RAM 22, a storage section 23, an imaging section 24, a light emitting section 25, a display section 26, an operating section 27, a communication section 28, and the like, and each of these sections is connected by a bus 29.

The CPU 21 is a processor that controls the operation of the second camera 20 by reading and executing a program 231 stored in the storage section 23 and performing various arithmetic processing. The second camera 20 may have a plurality of processors (for example, a plurality of CPUs), and the plurality of processes executed by the CPU 21 of the present embodiment may be executed by the plurality of processors. In this case, the plurality of processors may participate in a common process, or the plurality of processors may independently execute different processes in parallel. When capturing an image of a patient, the CPU 21 causes the light emitting section 25 to emit light in accordance with settings and user operations and causes the imaging section 24 to perform imaging in accordance with user operations that instruct imaging, thereby generating image data of the medical image. Furthermore, the CPU 21 can add the patient ID of the captured patient to the generated medical image as supplementary information. The patient ID may be included, for example, in the EXIF information of the medical image. This patient ID is, for example, input by a doctor who operates the second camera 20 using the operating section 27 prior to imaging by the second camera 20.

Since the configurations of the RAM 22, storage section 23, display section 26, operating section 27, and communication section 28 are similar to the configurations of the RAM 12, storage section 13, display section 16, operating section 17, and communication section 18 of the first camera 10, detailed explanations are omitted.

The imaging section 24, like the imaging section 14 of the first camera 10, includes an optical system, an imaging element, an image processing section, and the like. However, as described above, the optical system of the imaging section 24 is designed so that the angle of view is larger than the angle of view of the first camera 10 (so that the second imaging range R2 is larger than the first imaging range R1).

The light emitting section 25 emits light for illuminating a subject in the direction of imaging by the imaging section 24. As mentioned above, the light emitting section 25 emits normal white light. The on/off of light emission by the light emitting section 25 is controlled by a control signal sent from the CPU 21.

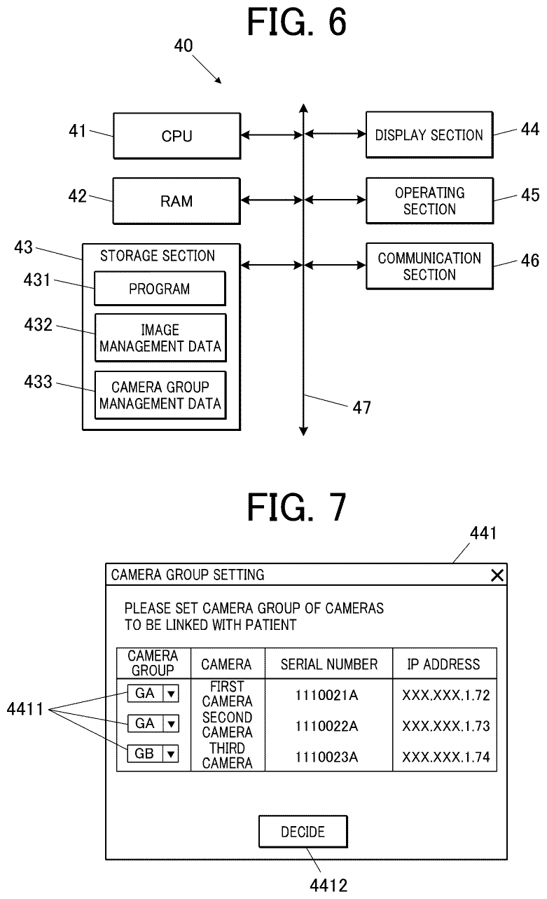

FIG. 6 is a block diagram showing the main functional configuration of the medical image management device 40.

The medical image management device 40 includes a CPU 41, a RAM 42, a storage section 43, a display section 44, an operating section 45, a communication section 46, and the like, and each of these sections is connected by a bus 47.

The CPU 41 is a processor (a processing unit, one or more processing units) that controls the operation of the medical image management device 40 by reading and executing a program 431 stored in the storage section 43 and performing various arithmetic processing. The medical image management device 40 may have a plurality of processors (for example, a plurality of CPUs), and the plurality of processes executed by the CPU 41 of the present embodiment may be executed by the plurality of processors. In this case, one or more processing units are configured by a plurality of processors. In this case, the plurality of processors may participate in a common process, or the plurality of processors may independently execute different processes in parallel.

The RAM 42 provides a working memory space for the CPU 41 and stores temporary data.

The storage section 43 is a non-transitory recording medium readable by the CPU 41 as a computer and stores a program 431 executed by the CPU 41, various data, and the like. The storage section 43 includes a non-volatile memory, for example, a hard disk drive (HDD) or a solid state drive (SSD). The program 431 is stored in the storage section 43 in the form of computer-readable program code. The data stored in the storage section 43 includes image management data 432 for managing medical images received from the first camera 10, the second camera 20, and the third camera 30, as well as camera group management data 433 for managing the camera group described below.

The display section 44 includes a display device such as a liquid crystal display. The display section 44 displays a medical image management screen, screens relating to various statuses of the medical image management system 1, and the like on the display screen of the display device in accordance with the image data and control signals transmitted from the CPU 41.

The operating section 45 includes a keyboard, a mouse, and a touch panel that is overlaid on the display screen. The operating section 45 detects operations on the keyboard, mouse, touch panel, and the like, and outputs operation signals according to the detection results to the CPU 41.

The communication section 46 is composed of, for example, a network card or a communication module, and transmits and receives data between the first camera 10, the second camera 20, and the third camera 30, or external devices such as a wireless LAN access point, in accordance with a predetermined communication standard.

### Operation of Medical Image Management System

Next, the operation of the medical image management system 1 will be described.

Here, a doctor who is an operator of the medical image management system 1, during an examination of a patient, captures the internal vulva using the first camera 10 and captures the patient's vulva using the second camera 20.

In the medical image management system 1 in the present embodiment, prior to imaging of a patient, a camera group (imaging apparatus group) consisting of a plurality of cameras to be used to image the same patient can be set. Additionally, a patient ID, which is a unique code assigned to each patient, can be registered in association with the camera group. By registering a patient ID in association with a camera group, a plurality of medical images (a plurality of images) captured by one or more cameras included in the camera group can be automatically linked with and managed by the patient ID. In other words, even if a patient ID has not been input to some of the cameras, medical images captured by all of the cameras included in the camera group can be automatically linked as medical images capturing the same patient.

The camera group can be set on a camera group setting screen 441 displayed on the display section 44 of the medical image management device 40.

FIG. 7 is a diagram showing the camera group setting screen 441.

The camera group setting screen 441 displays a list of the serial numbers and IP addresses of the first camera 10, the second camera 20, and the third camera 30 provided in the medical image management system 1. Furthermore, the camera group setting screen 441 is provided with a drop-down list 4411 for each camera for selecting the camera group to which the camera belongs. In FIG. 7, the drop-down list 4411 is selected so that the first camera 10 and the second camera 20 belong to the camera group GA, and the third camera 30 belongs to the camera group GB. Selecting the same camera group using the drop-down list 4411 for a plurality of cameras corresponds to “a user operation of selecting a plurality of cameras that make up a camera group.” When the decision button 4412 is selected, the camera group configuration is set to reflect the selection state of the drop-down list 4411 at that time and is registered in the camera group management data 433.

In place of the drop-down list 4411, check boxes for selecting the cameras that make up the camera group may be displayed.

In the following, the operation of the medical image management system 1 will be described using the camera group GA constituted by the first camera 10 and the second camera 20 as an example.

After the camera group GA is set, when a patient ID is input into one of the first camera 10 and the second camera 20 included in the camera group GA, the patient ID is transmitted from the one of the cameras to the medical image management device 40 and registered in association with the camera group GA.

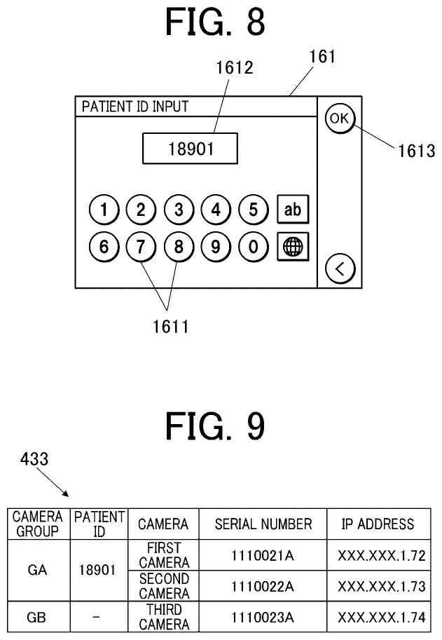

FIG. 8 is a diagram showing a patient ID input screen 161 for inputting a patient ID.

The patient ID input screen 161 is displayed on the display section 16 in response to a predetermined operation being performed on the operating section 17 of the first camera 10. The patient ID input screen 161 displays numeric keys 1611 for inputting a patient ID, a text box 1612 in which the input patient ID is displayed, and an OK button 1613. In the example shown in FIG. 8, a patient ID “18901” is input in a text box 1612 in response to operation of the numeric keys 1611. When the OK button 1613 is selected with the patient ID input in the text box 1612, the CPU 11 of the first camera 10 stores the patient ID in the storage section 13. In addition, the CPU 11 transmits the patient ID to the medical image management device 40 via the communication section 18.

The patient ID can also be input into the second camera 20 via a similar patient ID input screen and transmitted to the medical image management device 40. However, if the patient ID is input into one of the first camera 10 and the second camera 20, the patient ID is associated with the camera group GA, and therefore there is no need to input the patient ID into the other camera. In the following, an example will be described in which the patient ID “18901” is input only to the first camera 10.

When the CPU 41 of the medical image management device 40 receives the patient ID from the first camera 10, the CPU 41 registers the received patient ID in the camera group management data 433 in association with the camera group GA to which the first camera 10 belongs.

FIG. 9 is a diagram showing the camera group management data 433 in a state in which a patient ID is associated.

In the example shown in FIG. 9, the patient ID “18901” is registered in association with the camera group GA consisting of the first camera 10 and the second camera 20.

After the patient ID is registered for the camera group GA, when the operator, a doctor, captures the patient using the first camera 10 and the second camera 20, medical images are generated in each camera and transmitted to the medical image management device 40.

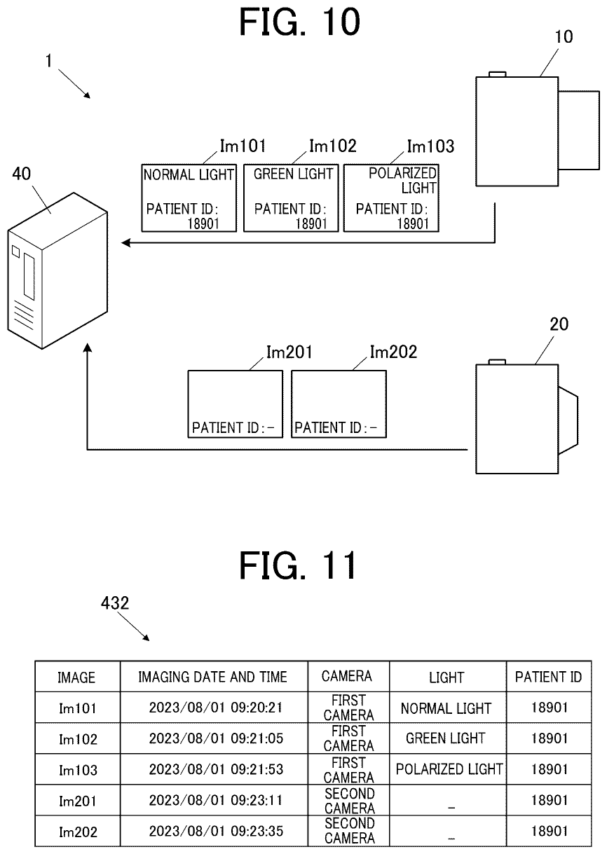

FIG. 10 is a diagram showing the operation of transmitting a medical image Im from the first camera 10 and the second camera 20.

Here, the first camera 10 generates first medical images Im101 to Im103 (first images) by the above-mentioned imaging, and the second camera 20 generates second medical images Im201, Im202 (second images) by the above-mentioned imaging. In the following, when referring to any one medical image captured by the first camera 10, it will be referred to as “first medical image Im1”, and when referring to any one medical image captured by the second camera 20, it will be referred to as “second medical image Im2”. Moreover, when referring to any one medical image captured by the first camera 10 or the second camera 20, it will be referred to as “medical image Im”.

The first medical image Im101 captured by the first camera 10 is captured using normal light 151, the first medical image Im102 is captured using green light 152, and the first medical image Im103 is captured using polarized light 153. Furthermore, the EXIF data of the first medical images Im101 to Im103 captured by the first camera 10 includes the patient ID “18901” input to the first camera 10.

On the other hand, the EXIF information of the second medical images Im201 and Im202 captured by the second camera 20 does not include the patient ID. This is because the patient ID has not been input to the second camera 20.

When imaging is performed by the first camera 10 and the second camera 20, the generated medical images Im are transmitted one by one to the medical image management device 40. After a plurality of medical images Im are captured by each camera, the plurality of medical images Im may be transmitted collectively from each camera.

The CPU 41 of the medical image management device 40 registers the received medical images Im in the image management data 432.

FIG. 11 is a diagram showing the contents of the image management data 432.

One data row of the image management data 432 corresponds to one medical image Im.

The “imaging date and time” in each data row is information on the imaging date and time extracted from the EXIF information of the medical image Im.

The “camera” is information about the imaging apparatus (the first camera 10 or the second camera 20) used to capture the medical image Im, extracted from the EXIF information of the medical image Im.

The “light” is information about the light used in imaging, extracted from the EXIF information of the medical image Im.

The “patient ID” is the patient ID of the patient who is the subject of the medical image Im. The patient ID is identified in the following manner and registered in association with each medical image Im.

When the CPU 41 receives a medical image Im, it refers to the camera group management data 433 shown in FIG. 9 and identifies the patient ID (here, “18901”) associated with the camera group GA to which the first camera 10 or second camera 20 that transmitted the medical image Im belongs. Then, the CPU 41 registers the identified patient ID in the image management data 432 in association with the received medical image Im. As a result, as shown in FIG. 11, the first medical images Im101 to Im103 received from the first camera 10 and the second medical images Im201 and Im202 received from the second camera 20 are all registered in association with the patient ID “18901”. In this way, even if the patient ID is not input in the second camera 20, the patient ID registered in association with the camera group GA is automatically registered in association with the medical image Im captured by the second camera 20. This makes it possible to link and manage the medical images Im received from the first camera 10 and the second camera 20 with and by the patient ID of the same patient.

For the first medical images Im101 to Im103 received from the first camera 10, the patient ID “18901” contained in the EXIF information of these medical images Im may be associated with each of the first medical images Im101 to Im103 and registered in the image management data 432.

When a medical image Im captured before the camera group GA was registered is received from the first camera 10 and/or the second camera 20, the CPU 41 does not associate the patient ID with the medical image Im. In other words, the CPU 41 performs association of the patient ID using the setting of the camera group described above only when the imaging time included in the EXIF information of the received medical image Im is later than the time when the camera group GA was registered.

In the above, an example was described in which medical images Im were transmitted from all of the cameras included in the camera group GA, that is, the first camera 10 and the second camera 20, but the transmission is not limited to this, and medical images Im may be transmitted from some of the cameras included in the camera group GA.

The contents of the image management data 432 shown in FIG. 11 may be displayed on the display section 44 of the medical image management device 40.

When switching the patient to be captured, for example, the patient ID of the new patient may be input into one of the first camera 10 and the second camera 20. Normally, the doctor who is the operator turns off the power of each camera when he or she has finished imaging of a patient. Thus, if the patient ID input screen 161 shown in FIG. 8 is displayed when the camera is turned on to image the next patient, the amount of operating work required to switch patients can be reduced.

The patient ID input when switching patients is transmitted to the medical image management device 40. If the latest patient ID received is different from the patient ID already registered for the camera group GA, the CPU 41 of the medical image management device 40 registers (updates) the latest patient ID to overwrite the patient ID already registered, in the camera group management data 433 in association with the camera group GA. When the CPU 41 receives a plurality of medical images Im captured by the first camera 10 and the second camera 20 after the latest patient ID is registered, the CPU 41 links the plurality of medical images Im by the latest patient ID. In this way, by inputting the switched patient ID into one of the cameras included in the camera group GA, it is possible to switch the patient ID to be linked to the medical images Im captured by each camera in the camera group GA (that is, it is possible to switch the patient).

Next, a camera group setting process and a medical image registration process executed by the CPU 41 of the medical image management device 40 to realize the above-mentioned operation of the medical image management system 1 will be described.

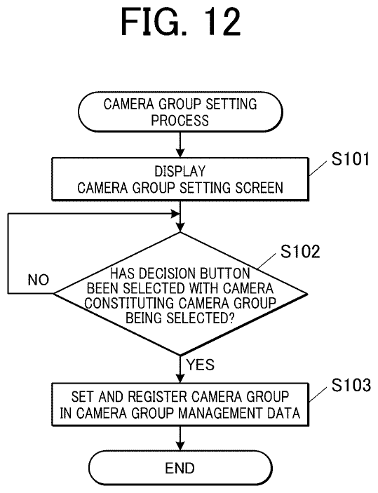

FIG. 12 is a flowchart showing a control procedure for the camera group setting process.

The camera group setting process is started when an operation for displaying the camera group setting screen 441 is performed on the medical image management device 40.

When the camera group setting process is started, the CPU 41 of the medical image management device 40 displays the camera group setting screen 441 of FIG. 7 on the display section 44 (step S101).

The CPU 41 repeatedly determines whether or not the decision button 4412 has been selected with a camera constituting the camera group being selected (step S102). When the CPU 41 determines that the operation has been performed (YES in step S102), it sets a camera group consisting of the selected cameras and registers it in the camera group management data 433 (step S103).

When step S103 ends, the CPU 41 ends the camera group setting process.

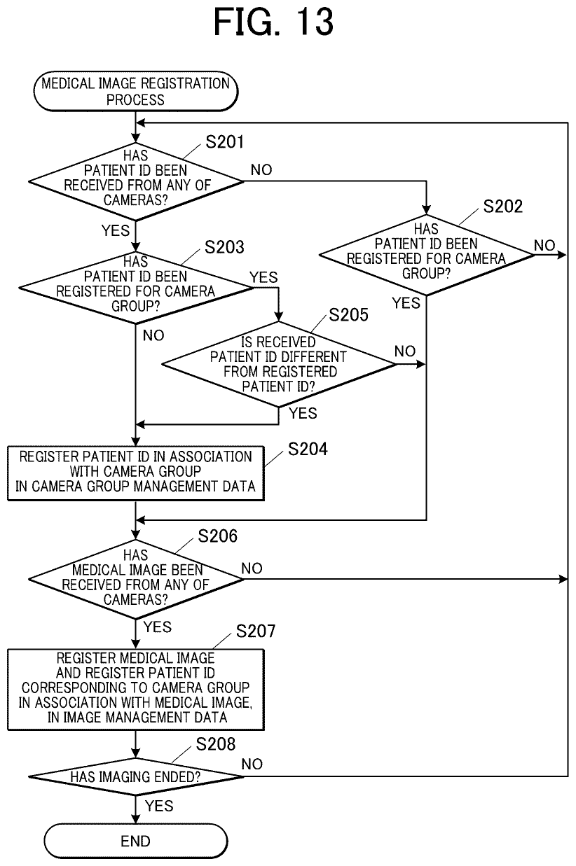

FIG. 13 is a flowchart showing a control procedure for the medical image registration process.

The medical image registration process is started when the camera group setting process is completed. The medical image registration process is executed for each set camera group. In the following, a medical image registration process corresponding to the camera group GA will be illustrated.

When the medical image registration process is started, the CPU 41 determines whether or not a patient ID has been received from any of the cameras included in the camera group GA (step S201). If determined that the patient ID has not been received (NO in step S201), the CPU 41 determines whether or not the patient ID has already been associated with the camera group GA and registered in the camera group management data 433 (step S202). If it is determined that the patient ID is not registered for the camera group GA (NO in step S202), the CPU 41 returns the process to step S201.

If it is determined that the patient ID has been received from any of the cameras (YES in step S201), the CPU 41 determines whether or not the patient ID has already been associated with the camera group GA and registered in the camera group management data 433 (step S203). If it is determined that the patient ID is not registered for the camera group GA (NO in step S203), the CPU 41 registers the received patient ID in association with the camera group GA in the camera group management data 433 (step S204). If it is determined in step S203 that the patient ID has already been registered for the camera group GA (YES in step S203), the CPU 41 determines whether or not the received patient ID is different from the registered patient ID (step S205). If the CPU 41 determines that the received patient ID is different from the registered patient ID (YES in step S205), it proceeds to step S204 and registers the received patient ID to overwrite the registered patient ID, in association with the camera group GA in the camera group management data 433.

Of the above, the flow proceeding in the order of steps S201, S203, and S204 corresponds to the process of first associating and registering a patient ID after the camera group GA is set.

Moreover, the flow proceeding in the order of step S201, S203, S205, and S204 corresponds to the process for switching the patient (patient ID).

When step S204 is completed, the CPU 41 executes step S206. In addition, if it is determined in step S202 that the patient ID has already been registered for the camera group GA (YES in step S202), or if it is determined in step S205 that the received patient ID is identical to the registered patient ID (NO in step S205), the CPU 41 executes step S206. In step S206, the CPU 41 determines whether or not a medical image Im has been received from any of the cameras included in the camera group GA. If it is determined that the medical image Im has not been received (NO in step S206), the CPU 41 returns the process to step S201. If it is determined that the medical image Im has been received (YES in step S206), the CPU 41 registers the received medical image Im in the image management data 432. Furthermore, the CPU 41 registers the patient ID corresponding to the camera group GA in association with the received medical image Im in the image management data 432 (step S207).

The CPU 41 determines whether or not imaging by the camera group GA has ended (step S208). For example, the CPU 41 determines that imaging has ended when the power of each camera included in the camera group GA is turned off. If the CPU 41 determines that the imaging has not ended (NO in step S208), the process returns to step S201. If the CPU 41 determines that the imaging has ended (NO in step S208), the CPU 41 ends the medical image registration process.

### Modification Example

Next, a modification example of the first embodiment will be described. In the following, the differences from the first embodiment will be explained, and the points in common with the first embodiment will not be explained.

In the above-mentioned first embodiment, the patient ID to be associated with the medical image Im was switched by inputting the patient ID in the first camera 10 or the second camera 20, but in the modification example, the patient ID can be switched by imaging an identification sign 50 from which the patient ID (or the patient) can be identified with the first camera 10 or the second camera 20.

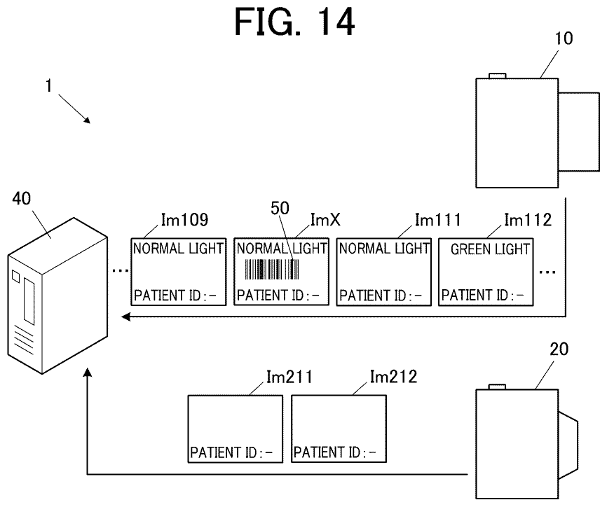

FIG. 14 is a diagram showing a transmission operation of a medical image Im according to a modification example.

After completing imaging of a patient, the doctor, who is the operator, performs imaging of a predetermined identification sign 50 from which the patient ID of the next patient can be identified using the first camera 10 or the second camera 20 before imaging of the next patient. The identification sign 50 may be an image, such as a barcode or two-dimensional code, that can be decoded to obtain the patient ID. Furthermore, the identification sign 50 may be a code (a figure, a symbol, a character, or a combination of these, and the like) that is associated one-to-one with the patient ID in predetermined table data. Additionally, the identification sign 50 may be the patient ID itself. In the example shown in FIG. 14, a medical image Im109 capturing a patient is generated by the first camera 10, and then an identification sign image ImX capturing an identification sign 50 is generated. Thereafter, first medical images Im111, Im112 and second medical images Im211, Im212 capturing the next patient are generated.

The series of medical images Im and the identification sign image ImX are transmitted to the medical image management device 40 and registered in the image management data 432. The identification sign image ImX is used in the medical image registration process to register the patient ID for the camera group GA and to switch patients.

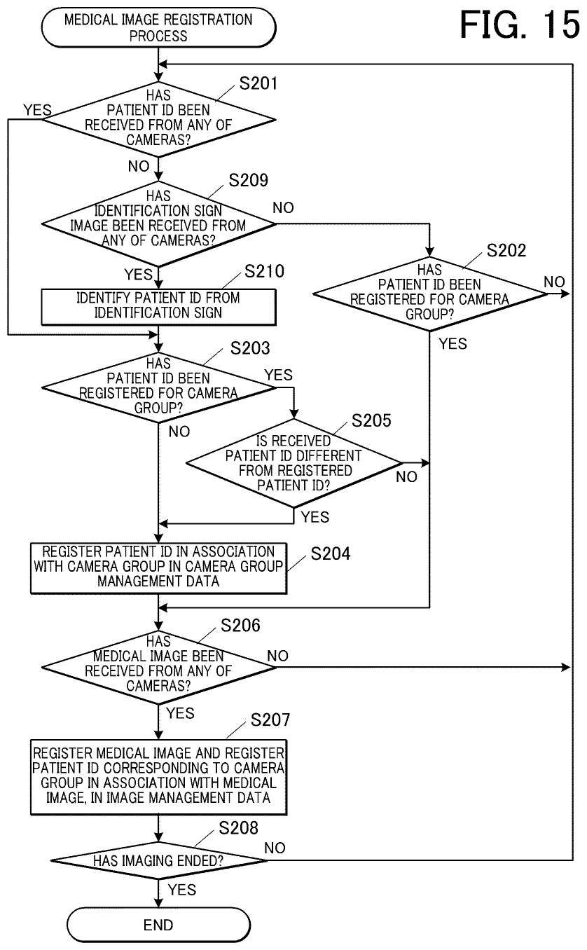

FIG. 15 is a flowchart showing a control procedure of the medical image registration process according to the modification example.

The flowchart in FIG. 15 is obtained by adding steps S209 and S210 to the flowchart in FIG. 13. In the following, description of the processes common to the flowchart of FIG. 13 will be omitted.

If the CPU 41 of the medical image management device 40 determines that a patient ID has not been received from a camera included in the camera group GA (NO in step S201), the CPU 41 determines whether or not an identification sign image ImX has been received from any of the cameras (step S209). For example, the CPU 41 performs a known image recognition process for extracting the identification sign 50 to the image data of the received image. If the identification sign 50 is extracted, the CPU 41 determines that the identification sign image ImX has been received. If it is determined that the identification sign image ImX has not been received (NO in step S209), the CPU 41 shifts the process to step S202. When it is determined that the identification sign image ImX has been received (YES in step S209), the CPU 41 identifies the patient ID from the extracted identification sign 50 (step S210). For example, when the identification sign 50 is a barcode or a two-dimensional code, the CPU 41 performs a known decoding process or the like on the identification sign 50 to identify the patient ID corresponding to the identification sign 50. Furthermore, when the identification sign 50 is the above-mentioned code, the CPU 41 identifies the patient ID corresponding to the identification sign 50 by referring to predetermined table data. When step S210 ends, the CPU 41 advances the process to step S203. The subsequent processing is similar to that in FIG. 13.

According to the medical image registration process of the modification example, when the identification sign image ImX is received (YES in step S209) and the patient ID is not registered in association with the camera group GA (NO in step S203), the patient ID identified from the identification sign 50 of the identification sign image ImX is registered in association with the camera group GA (step S204). The flow proceeding in this order of steps S209, S210, S203, and S204 corresponds to the process of first associating and registering a patient ID after the camera group GA is set.

In addition, if the patient ID has already been registered in the camera group GA (YES in step S203) and the patient ID identified from the identification sign 50 of the identification sign image ImX (the received patient ID) is different from the registered patient ID (YES in step S205), the patient ID identified from the identification sign 50 is associated with the camera group GA and overwritten and registered (step S204). The flow proceeding in this order of steps S209, S210, S203, S205, and S204 corresponds to the process when switching the patient (patient ID).

In the modification example, step S201 of receiving the patient ID from the first camera 10 or the second camera 20 may be omitted. That is, only the patient ID identified from the identification sign 50 of the identification sign image ImX may be used for managing the medical image Im.

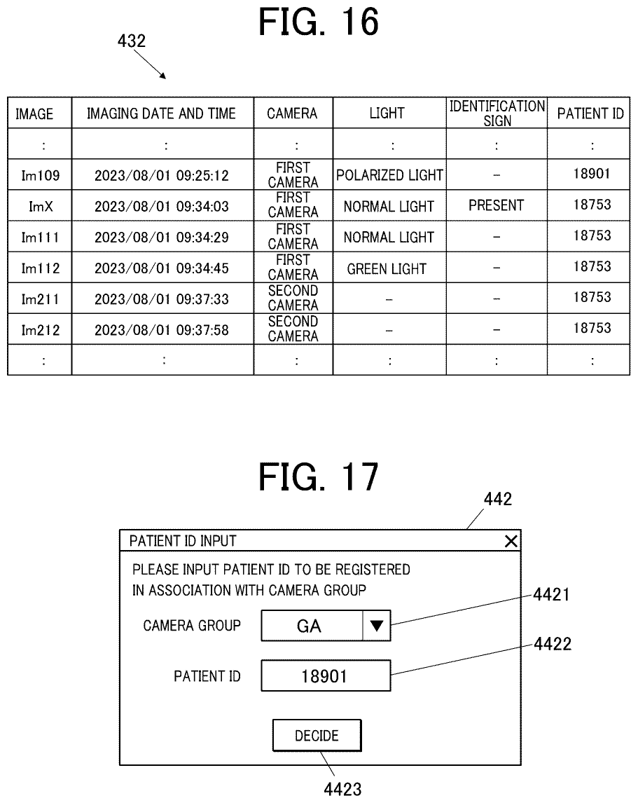

FIG. 16 is a diagram showing the contents of the image management data 432 relating to the modification example.

By executing the medical image registration process shown in FIG. 15, the image management data 432 shown in FIG. 16 is generated. In the image management data 432 of the modification example, a data item for “identification sign” is provided, and if the image contains an identification sign 50 (if the image is an identification sign image ImX), the “identification sign” is marked as “present.” When the patient ID (here, “18753”) identified from the identification sign 50 of this identification sign image ImX is registered to overwrite the registered ID in association with the camera group GA in the camera group management data 433, the patient ID is registered in association with the first medical images Im111, Im112 and the second medical images Im211, Im212 captured subsequently. As a result, when the patient is switched to a new patient, a plurality of medical images Im can be linked with and managed by the patient ID of the new patient after the switching.

### Effects

As described above, the program 431 according to the first embodiment causes the CPU 41 of the medical image management device 40 to execute the following processes in response to a user's operation of selecting a plurality of cameras (the first camera 10 and the second camera 20): setting a group camera GA including the plurality of cameras; registering a patient ID from which a patient can be identified in association with the camera group GA; acquiring a plurality of medical images Im captured by respective ones of the plurality of cameras included in the camera group GA; and linking the acquired a plurality of medical images Im with the patient ID. As a result, even when imaging is performed by switching between a plurality of cameras, the medical images Im captured by the respective cameras can be appropriately linked as medical images Im of the same patient. Therefore, it is possible to easily manage medical images Im when a plurality of cameras are used. This reduces the burden on the operator and shortens the examination time.

In addition, the program 431 causes the CPU 41 to execute a process of acquiring a patient ID from a camera included in the camera group GA and registering the patient ID in association with the camera group GA when the patient ID is input by a user to the camera. This makes it possible to link the medical images Im captured by the respective cameras included in the camera group GA by the same patient ID by a simple operation of inputting the patient ID to one of the plurality of cameras. This reduces the burden on the operator compared to the conventional method in which a patient ID had to be input to each camera used. In addition, it is possible to suppress the occurrence of problems such as forgetting to input the patient ID or inputting an incorrect ID, which results in inappropriate linking of the medical images Im.

In addition, when the latest patient ID acquired from any camera included in the camera group GA is different from the patient ID already registered in association with the camera group GA, the program 431 causes the CPU 41 to execute the following processes: registering the latest patient ID t overwrite the patient ID already registered in association with the camera group GA; acquiring a plurality of medical images Im captured by a camera included in the camera group GA after registration of the latest patient ID; and linking the acquired a plurality of medical images Im by the latest patient ID. This makes it possible to switch the patient ID (switch the patient) used for linking the medical images Im by a simple operation of inputting the patient ID to any of the plurality of cameras.

In addition, in the modification example, when the program 431 acquires an identification sign image ImX that captures an identification sign 50 from which a patient ID can be identified from a camera included in the camera group GA, the program 431 causes the CPU 41 to execute a process of identifying the patient ID based on the identification sign 50 and registering the patient ID in association with the camera group GA. According to this method, since there is no need to manually input the patient ID, the burden on the operator can be further reduced.

In addition, in the modification example, when the latest patient ID identified from the acquired identification sign image ImX is different from the patient ID already registered in association with the camera group GA, the program 431 causes the CPU 41 to execute the following processes: registering the latest patient ID to overwrite the patient ID already registered in association with the camera group GA; acquiring a plurality of medical images Im captured after imaging of the identification sign image ImX by a camera included in the camera group GA; and linking the acquired a plurality of medical images Im by the latest patient ID. This makes it possible to switch the patient ID (switch the patient) used for linking the medical images Im by a simple operation of capturing the identification sign 50 with any of the plurality of cameras.

The medical image management system 1 includes: a first camera 10 having a first imaging range R1 and capturing a first medical image Im1 relating to a first site of the patient; and a second camera 20 having a second imaging range R2 different from the first imaging range R1 and capturing a second medical image Im2 relating to a second site different from the first site of the patient. In addition, the program 431 causes the CPU 41 to execute the following processes: acquiring at least one first medical image Im1 and at least one second medical image Im2 captured by the first camera 10 and the second camera 20 included in the camera group GA; and linking the acquired at least one first medical image Im1 and at least one second medical image Im2 with the patient ID. Thus, when it is necessary to switch the imaging apparatus used between the first camera 10 and the second camera 20 depending on the site to be captured, the medical images Im captured by each camera can be appropriately linked as medical images Im of the same patient.

In addition, the medical image management device 40 according to the first embodiment includes the CPU 41, which sets the camera group GA consisting of a plurality of cameras in response to a user's operation of selecting the plurality of cameras for capturing the medical images Im, registers the patient ID from which the patient can be identified in association with the camera group GA, acquires the plurality of medical images Im captured by respective ones of the plurality of cameras included in the camera group GA, and links the acquired a plurality of medical images Im with the patient ID. This makes it possible to easily manage the medical images Im when the plurality of cameras are used.

In addition, the image management method according to the second embodiment sets the camera group GA consisting of a plurality of cameras in response to a user's operation to select the plurality of cameras for capturing medical images Im, registers the patient ID from which the patient can be identified in association with the camera group GA, acquires the plurality of medical images Im captured by the cameras included in the camera group GA, and links the acquired a plurality of medical images Im with the patient ID. This makes it possible to easily manage the medical images Im when the plurality of cameras are used.

### Second Embodiment

Next, the second embodiment will be described. Below, differences from the first embodiment will be explained, and the explanation of the points in common with the first embodiment will be omitted.

In the first embodiment, the patient ID is input in the first camera 10 or the second camera 20, but in the second embodiment, the patient ID is input by a user's operation in the medical image management device 40. In the present embodiment, for example, when the decision button 4412 is selected on the camera group setting screen 441 shown in FIG. 7 to set the camera group, a patient ID input screen 442 for inputting a patient ID to be registered in association with the camera group is displayed on the display section 44.

FIG. 17 is a diagram showing the patient ID input screen 442.

The patient ID input screen 442 displays a drop-down list 4421 for selecting a camera group for which the patient ID is to be registered, a text box 4422 for inputting the patient ID, and a decision button 4423. When a camera group is selected from the drop-down list 4421 and the decision button 4423 is selected with a patient ID input in the text box 4422, the input patient ID is registered in association with the selected camera group in the camera group management data 433, as shown in FIG. 9.

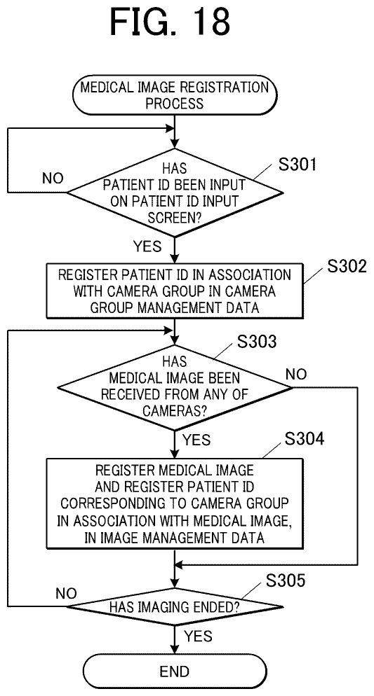

FIG. 18 is a flowchart showing a control procedure of the medical image registration process according to the second embodiment.

When the medical image registration process is started, the CPU 41 repeatedly determines whether or not an operation to input a patient ID has been performed on the patient ID input screen 442 (step S301). When it is determined that the operation has been performed (YES in step S301), the CPU 41 registers the input patient ID in association with the selected camera group GA in the camera group management data 433 (step S302). The CPU 41 determines whether or not a medical image Im has been received from any of the cameras included in the camera group GA (step S303). When it is determined that the medical image Im has been received (YES in step S303), the CPU 41 registers the received medical image Im in the image management data 432. Furthermore, the CPU 41 registers the patient ID corresponding to the camera group GA in association with the received medical image Im in the image management data 432 (step S304). When step S304 is completed, or when it is determined that the medical image Im has not been received (NO in step S303), the CPU 41 determines whether or not the imaging by the camera group GA has ended (step S305). If the CPU 41 determines that the imaging has not ended (NO in step S305), the CPU 41 returns the process to step S303; if the CPU 41 determines that the imaging has ended (NO in step S305), the CPU 41 ends the medical image registration process.

### Effect

As described above, the program 431 according to the second embodiment causes the CPU 41 of the medical image management device 40 to execute a process of registering a patient ID in association with the camera group GA when the patient ID is input by a user in the medical image management device 40. This makes it possible to link medical images Im captured by respective cameras included in the camera group GA by the same patient ID by a simple operation of inputting the patient ID to the medical image management device 40. This reduces the burden on the operator compared to the conventional method in which a patient ID had to be input to each camera used. In addition, it is possible to suppress the occurrence of problems such as forgetting to input the patient ID or inputting an incorrect ID, which results in inappropriate linking of the medical image Im.

### Others

The present disclosure is not limited to the above-described embodiments, and various modifications are possible. For example, in the above embodiments, the medical image management system 1 is applied to imaging of the internal vulva and the vulva in obstetrics and gynecology, but the present disclosure is not limited to this. The medical image management system 1 can be used for any purpose in which a plurality of medical images Im are captured by a plurality of imaging apparatuses.

For example, when the inside of a patient's mouth (the inside of the teeth and the throat) is captured by a first imaging apparatus, and the surface of the patient's teeth or part of the face is captured by a second imaging apparatus, the first medical image captured by the first imaging apparatus and the second medical image captured by the second imaging apparatus may be linked using the above-mentioned embodiment methods.

Furthermore, the imaging ranges of the plurality of imaging apparatuses are not limited to those different from each other. For example, in a configuration in which two imaging apparatuses with the same specifications are used, one as a main imaging apparatus and the other as a sub imaging apparatus, and the main imaging apparatus and the sub imaging apparatus are switched to be used as needed, medical images captured by these two imaging apparatuses may be linked using the above-mentioned embodiment methods.

Furthermore, the processes executed by the CPU 41 of the medical image management device 40 in the above embodiments may be executed by the CPU 11 of the first camera 10 (or the CPU 21 of the second camera 20). In this case, the first camera 10 (or the second camera 20) corresponds to the “medical information management device”, and the CPU 11 (or the CPU 21) corresponds to the “processing unit”.

In addition, in the above embodiments, a medical image Im is exemplified as an image to be managed, but the image to be managed is not limited to this and may be any image other than a medical image Im.

In the above description, an example has been given in which the HDD or SSD of the storage section 43 is used as a computer-readable medium for the program according to the present disclosure, but the computer-readable medium is not limited to this example. As other computer-readable media, information recording media such as flash memory and CD-ROM can be used. In addition, carrier waves are also applied to the present disclosure as a medium for providing data of the program according to the present disclosure via a communication line.

Furthermore, it goes without saying that the detailed configuration and detailed operation of each component of the medical image management system 1 in the above embodiment can be modified as appropriate without departing from the spirit of the present disclosure.

Although the embodiments of the present disclosure have been described, the scope of the present disclosure is not limited to the above-described embodiments but includes the scope of the claims and their equivalents.

1. An image management method executed by a computer, the method comprising:
setting an imaging apparatus group including a plurality of imaging apparatuses in response to a user's operation of selecting the plurality of imaging apparatuses;
registering identification information capable of identifying a subject in association with the imaging apparatus group;
acquiring a plurality of images captured by respective ones of the plurality of imaging apparatuses included in the imaging apparatus group; and
linking the acquired plurality of images with the identification information.

2. The image management method according to claim 1, wherein, in response to the identification information being input by a user in an imaging apparatus included in the imaging apparatus group, the identification information is acquired from the imaging apparatus in which the identification information is input, and registered in association with the imaging apparatus group.

3. The image management method according to claim 2, wherein
latest identification information acquired from an imaging apparatus included in the imaging apparatus group is different from the identification information already registered in association with the imaging apparatus group,
the latest identification information is registered to overwrite the identification information already registered, in association with the imaging apparatus group,
a plurality of images captured by an imaging apparatus included in the imaging apparatus group after registration of the latest identification information are acquired, and
the acquired plurality of images are linked by the latest identification information.

4. The image management method according to claim 1, wherein, in response to an identification sign image being acquired from an imaging apparatus included in the imaging apparatus group, the identification sign imaging an identification sign capable of identifying the identification information, the identification information is identified based on the identification sign, and the identification information is registered in association with the imaging apparatus group.

5. The image management method according to claim 4, wherein, latest identification information identified from the acquired identification sign image is different from the identification information already registered in association with the imaging apparatus group,
the latest identification information is registered to overwrite the identification information already registered, in association with the imaging apparatus group,
a plurality of images captured by an imaging apparatus included in the imaging apparatus group after capturing of the identification sign image are acquired, and
the acquired plurality of images are linked by the latest identification information.

6. The image management method according to claim 1, wherein, in response to the identification information being input by a user, the identification information is registered in association with the imaging apparatus group.

7. The image management method according to claim 1, wherein
the plurality of imaging apparatuses include a first imaging apparatus having a first imaging range and capturing a first image relating to a first site of the subject, and a second imaging apparatus having a second imaging range different from the first imaging range and capturing a second image relating to a second site different from the first site of the subject, and
in the image management method, the computer
acquires at least one of the first image and at least one of the second image respectively captured by the first imaging apparatus and the second imaging apparatus included in the imaging apparatus group, and
links, with the identification information, the at least one of the first image and the at least one of the second image that are acquired.

8. An image management device comprising a processor that
sets an imaging apparatus group including a plurality of imaging apparatuses in response to a user's operation of selecting the plurality of imaging apparatuses,
registers identification information capable of identifying a subject in association with the imaging apparatus group,
acquires a plurality of images captured by respective ones of the plurality of imaging apparatuses included in the imaging apparatus group; and
links the acquired plurality of images with the identification information.
